(19) United States
(12) Patent Application Publication
(10) Pub. No.: US 2010/0196580 A1
(43) Pub. Date: Aug. 5, 2010

(54) FAD-2 MUTANTS AND HIGH OLEIC ACID PLANTS

(75) Inventors: Jean-Pierre Despeghel, Ingre (FR); Kunsheng Wu, Ballwin, MO (US); Heinrich Busch, Delbruck (DE)

Correspondence Address: SENNIGER POWERS LLP, 100 NORTH BROADWAY, 17TH FLOOR, ST LOUIS, MO, 63102, US

(73) Assignees: MONSANTO SAS, Lyon, FR (FR); DEUTSCHE SAATVEREDELUNG AG, Lippstadt, DE (DE)

(21) Appl. No.: 12/522,798

(22) PCT Filed: Jan. 11, 2008
(86) PCT No.: PCT/EP08/50307

§ 371 Date: Apr. 12, 2010

(30) Foreign Application Priority Data
Jan. 11, 2007 (EP) 07290043.4

(51) Int. Cl. A23D 9/00; C07H 21/04; C12N 9/02; C12N 15/63; C12N 5/10; A01H 5/00; A01H 1/00; C11B 1/00; A23L 1/212

(52) U.S. Cl. 426/601; 536/23.2; 435/189; 435/320.1; 435/419; 800/298; 800/322; 800/312; 800/320.1; 800/314; 800/281; 554/8; 426/615

(57) ABSTRACT

The present invention relates to plants, seeds and products derived thereof, in particular to Brassica plants, seeds products derived thereof, that have mutant sequences conferring high oleic acid profile on the seed oil. More particularly, the invention relates to mutant delta-12 fatty acid desaturase sequences, also referred to herein as FAD2 sequences, in such plants which confer high oleic acid profile on the seed oil.

### FIELD OF THE INVENTION

The present invention relates to plants, seeds and products derived thereof, in particular to Brassica plants, seeds products derived thereof, that have mutant sequences conferring high oleic acid profile to the seed oil.

More particularly, the invention relates to mutant delta-12 fatty acid desaturase sequences, also referred to herein as FAD2 sequences, in such plants which confer high oleic acid profile on the seed oil.

### BACKGROUND

Delta-12 fatty acid desaturase (also known as oleic desaturase or oleate desaturase) is involved in the enzymatic conversion of oleic acid to linoleic acid.

Varieties with high level of oleic acid (possibly combined with low level of linolenic acid) are sought for many different applications (food applications, health applications, biodiesel applications and many others).

Mutant seeds providing an oil exhibiting a high oleic acid content (oleic acid content higher that 70 wt. % based upon the total weight of fatty acids present in the oil) previously reported in the literature had very poor agronomic value and/or bad root characteristics, and/or very low yield capacity and/Or bad germination capacity and/or an oleic acid content stability problem across environments.

There is still a need for material having stable, high oleic acid content (possibly combined with stable low linolenic acid content) across locations and across years, with also good germination, good agronomic performances and with normal oilseed rape morphology. In particular, the plants should have no fasciation and should have normal root development.

### SUMMARY OF THE INVENTION

The present invention relates to a nucleic acid molecule comprising (or consisting of) a nucleic acid sequence encoding a delta-12 oleate desaturase (FAD2) protein, said FAD2 protein having an amino acid substitution at position 216 relative to a wild-type FAD2 protein.

Preferably, said FAD2 protein is a Brassica FAD2 protein, more particularly a Brassica napus FAD2 protein.

Preferably, said substituted amino acid at position 216 is a serine (replacing the proline at position 216 of a wild-type FAD2 protein).

A preferred nucleic acid molecule of the invention comprises (or consists of) a nucleic acid of SEQ ID NO 1, 2, or 3, its complementary form or its RNA form.

A nucleic acid molecule of the invention can comprise or consist of a nucleotide sequence having at least 80%, preferably at least 85%, more preferably at least 90% and even more preferably at least 95%, 96%, 97%, 98% or 99% identity with SEQ ID NO 1, 2 or 3, or with the complementary form or RNA form thereof, encoding a FAD2 protein having an amino acid substitution at position 216 relative to a wild-type FAD2 protein.

More particularly, said wild-type FAD2 protein comprises (or consists of) an amino acid sequence of SEQ ID NO 11 or 15.

Also object of the present invention is a fragment of at least 10, 15, 20, 25, 30, 40, 50, 100 or more nucleotides of a nucleic acid molecule according to the invention, said fragment comprising the mutated codon corresponding to said amino acid substitution at position 216.

Said fragments can be used as primers, probes and/or selectable markers.

Any of the nucleic acid molecules of the invention can be used in a method of marker assisted selection of plants, preferably of Brassica species, more preferably of Brassica napus varieties, also object of the present invention.

Another object of the present invention is an assay kit which can comprise a first container containing any of the nucleic acid molecules of the invention.

Another object of the present invention is a FAD2 protein having an amino acid substitution at position 216, or corresponding to position 216, relative to a wild-type FAD2 protein such as the wild-type FAD2 protein represented by the amino acid sequence of SEQ ID NO 11 or 15.

A preferred FAD2 protein of the invention comprises (or consists of) an amino acid sequence of SEQ ID NO 4.

Another object of the present invention is a vector comprising a nucleic acid molecule encoding a mutant FAD2 protein according to the invention.

Another object of the present invention is a host cell comprising a vector of the invention and/or a nucleic acid sequence encoding a mutant FAD2 protein according to the invention.

Another object of the present invention is a plant stably transformed with a vector of the invention.

A plant to be transformed can be selected from the group consisting of oil producing crops, more particularly, from sunflowers, soybeans, cottons, corns and/or rapeseeds.

Another object of the present invention is a plant or a plant part or a seed containing a nucleotide sequence encoding a FAD-2 protein having an amino acid substitution at or corresponding to position 216 relative to a wild-type FAD-2 protein.

More particularly, a plant or a plant part or a seed according to the invention contains (or expresses) a FAD-2 protein having an amino acid substitution at or corresponding to position 216 relative to a wild-type FAD-2 protein.

Preferably, said substituted amino acid at or corresponding to position 216 is a serine (replacing a proline at position 216 of a wild-type FAD2 protein).

A plant or a plant part or a seed according to the invention can be obtained by a mutagenesis treatment, more particularly by an EMS treatment.

Progenies derived from said plant or plant part or seed are also objects of the invention.

Another object of the present invention is a vegetable oil obtained from seeds of the invention, said oil comprising more than (about) 80%, 81%, 82%, 83%, 84%, 85%, 86% or 87% of oleic acid based upon the total weight of the fatty acids present in the rapeseed oil.

Preferably, said oil further comprises less than (about) 4%, 3.5%, 3%, 2%, 1% or 0.5% of linolenic acid.

The invention also relates to food or feed products containing and/or prepared with a plant, a plant part, a seed and/or a vegetable oil according to the invention.

A method of enhancing the oleic acid content in a plant can comprise the step of transforming a plant with a vector of the invention.

Alternatively, a method of producing high oleic plant lines can comprise:

(a) crossing a first plant of the invention with a second plant,
(b) obtaining seeds from the cross of step (a),
(c) growing fertile plants from such seeds,
(d) obtaining progeny seeds from the plants of step(c), and
(e) identifying those seeds among the progeny that have high oleic acid content.

Alternatively, a method of producing high oleic plant lines can comprise:
(a) inducing mutagenesis in at least some cells from a plant, more particularly of a Brassica plant, and preferably of a Brassica napus variety that has a oleic acid content of less than 70%;
(b) regenerating plants from at least one of said mutagenized cells;
(c) selecting regenerated plants which have any of the nucleic acid sequences of the invention and/or which expresses a FAD2 protein according to the invention; and
(d) deriving further generations of plants from said regenerated plants.

### BRIEF DESCRIPTION OF THE FIGURE

FIG. 1 corresponds to the list of sequences of the present invention.

### DETAILED DESCRIPTION OF THE INVENTION

The present invention relates to plants, more particularly to Brassica plants, preferably to Brassica napus varieties, which have been created for providing an oil having a oleic acid content higher than 80 wt. %, based upon the total weight of fatty acids present in the oil.

More particularly, a plant of the invention has at least one mutated FAD2 gene of the invention.

Preferably, said mutated FAD2 gene confers very high oleic acid content (i.e. a oleic acid content higher than 80 wt. %, based upon the total weight of fatty acids present in the oil) in seeds of said plants and in oil extracted from said seeds.

The present invention relates also to any part or any product of said plant bearing said at least one mutated FAD2 gene.

In the context of the present invention, a part or product of a plant is meant to encompass a leaf, cotyledon, stem, petiole, stalk, seed or any other tissue or fragment of tissue of said plant.

The present invention relates also to any progeny of said plant bearing said at least one mutated FAD2 gene of the invention.

In the context of the present invention, the term “progeny” refers to direct and indirect descendants, offspring and derivatives of a plant or plants of the invention and includes the first, second, third and/or subsequent generations, which may be produced by self crossing, crossing with plants with the same or different genotypes, and may be modified by range of suitable genetic engineering techniques.

The present invention also relates to said mutated FAD2 genes that confer high oleic acid content in seeds when present in a plant.

In particular, the invention relates to novel isolated nucleic acid molecules that encode novel variant forms of FAD2 protein having a substituted amino acid at position 216 (or corresponding to position 216) relative to a wild-type FAD2 protein, such as the wild-type FAD2 protein represented by SEQ ID NO 11 or more particularly by SEQ ID NO 15.

An isolated nucleic acid molecule of the invention contains said at least one mutation, resulting in a substitution, preferably a substitution of serine for proline, at (or corresponding to) position 216 relative to a wild-type FAD2 protein, such as the wild-type FAD2 protein represented by SEQ ID NO 11, or more particularly by SEQ ID NO 15.

Said mutation(s) alter(s) the functionality of the resulting FAD2 gene product, whereby the level of oleic acid is modified, preferably increased, in plant expressing the mutant sequence(s), compared to the corresponding level in plant expressing the wild-type sequence(s).

In the framework of the present invention, except if otherwise specified, the term “at position 216” is to be understood as designating the amino acid position 216 in a wild-type FAD2 protein represented by SEQ ID NO 11 or more particularly by SEQ ID NO 15, but also as referring to the amino acid corresponding to said position in a wild-type FAD2 protein that would have a different amino acid sequence due to deletions or additional amino acids in the polypeptide.

The term “corresponding to position” as used herein means that a position is not only determined by the number of the preceding amino acids. The position of a given amino acid in accordance with the present invention may vary due to deletions or additional amino acids in the polypeptide. Thus, under a “corresponding position” in accordance with the present invention it is to be understood that the amino acid(s) referred to may differ in the indicated number but still has (have) similar neighbouring amino acids in the linear sequence.

In one aspect, a nucleic acid molecule of the invention encodes a FAD2 protein having a substitution of a non-ionisable polar amino acid for a proline at position 216 relative to a wild type FAD2 protein, such as the wild type FAD2 protein represented by the amino acid sequence of SEQ ID NO 11 or more particularly of SEQ ID NO 15.

Said non-ionisable polar amino acid can be threonine, asparagine, glutamine, cysteine, tyrosine or serine. Preferably, said non-ionisable polar amino acid is serine.

A nucleic acid molecule of the invention can be derived (be generated, or be designed) from a nucleic acid molecule having a nucleic acid sequence of SEQ ID NO 5, 6, 8, 9, 10, 12, 13, 14 or 20.

Indeed, a nucleic acid molecule of the invention can comprise (or consist of) a nucleic acid sequence of SEQ ID NO 5, 6, 8, 9, 10, 12, 13, 14 or 20 wherein the codon encoding the amino acid at position 216 (or corresponding to position 216) of said wild-type FAD2 protein is mutated to encode an amino acid different from proline, or preferably to encode a non-ionisable polar amino acid (e.g. a threonine, asparagine, glutamine, cysteine, or a tyrosine), or more preferably to encode a serine.

Preferably, a nucleic acid molecule of the invention encodes a FAD2 protein having a substitution of a serine for a proline at position 216 (or corresponding to position 216) relative to a wild type FAD2 protein, such as the wild type FAD2 protein represented by the amino acid sequence of SEQ ID NO 11 or 15.

A preferred nucleic acid molecule of the invention comprises (or consists of) a nucleic acid sequence of SEQ ID NO 1, 2 or 3.

A nucleic acid molecule of the invention can be derived (generated, or designed) from a nucleic acid molecule having a nucleic acid sequence of SEQ ID NO 1, 2 or 3.

Indeed, a nucleic acid molecule of the invention can comprise (or consist of) a nucleic acid sequence of SEQ ID NO 1, 2 or 3, wherein the codon TCC encoding said serine at position 216 is mutated to encode an amino acid different from proline, or preferably to encode a non-ionisable polar amino acid (e.g. a threonine, asparagine, glutamine, cysteine, or a tyrosine), or more preferably to encode a serine.

Also object of the present invention is a fragment of at least 10, 15, 20, 25, 30, 40, 50, 100 or more nucleotides of a nucleic acid molecule according to the invention, said fragment comprising the mutated codon corresponding to said amino acid substitution at position 216.

In another aspect, a nucleic acid molecule of the invention can encode a FAD2 protein having a deletion at position 216 relative to a wild type FAD2 protein, such as a wild type FAD2 protein represented by the amino acid sequence of SEQ ID NO 11 or 15.

More particularly, a nucleic acid molecule of the invention can encode a FAD2 protein having a proline deleted at position 216 relative to a wild type FAD2 protein represented by the amino acid sequence of SEQ ID NO 11 or 15.

A nucleic acid molecule of the invention can be derived (generated, or designed) from a nucleic acid molecule having a nucleic acid sequence of SEQ ID NO 1, 2, 3, 5, 6, 8, 9, 10, 12, 13, 14 or 20.

Indeed, a nucleic acid molecule of the invention can comprise (or consist of) a nucleic acid sequence of SEQ

ID NO 1, 2, 3, 5, 6, 8, 9, 10, 12, 13, 14 or 20 wherein the codon encoding the amino acid at position 216 (or corresponding to position 216) is deleted.

Also object of the present invention is a fragment of at least 10, 15, 20, 25, 30, 40, 50, 100 or more nucleotides of a nucleic acid molecule according to the invention, said fragment comprising the deletion of said codon encoding said amino acid at position 216 (or corresponding to said position 216).

It will be appreciated by the skilled person that the nucleic acid sequences of SEQ ID NO 1-3, 5, 6, 8-10, 12-14, and 20 are not the only sequences that can be used to provide a FAD2 protein of the invention. Also contemplated are any nucleic acid molecules having different sequences but which, because of the degeneracy of the genetic code, encode a FAD2 protein comprising a substitution of an amino acid at position 216 (or corresponding to position 216) relative to the wild-type amino acid sequence, such as the wild-type FAD2 protein represented by SEQ ID NO 11 or 15.

In particular, a nucleic acid molecule of the invention can comprise (or consist of) a nucleotide sequence having at least 80%, preferably at least 85%, more preferably at least 90% and even more preferably at least 95%, 96%, 97%, 98% or 99% identity with any of SEQ ID NO 1-3, 5, 6, 8-10, 12-14, 20, or with the complementary form or RNA form thereof, encoding a FAD2 protein having an amino acid substitution at position 216 (or corresponding to position 216) relative to a wild-type FAD2 protein, such as the wild-type FAD2 protein represented by SEQ ID NO 11 or 15.

A nucleic acid molecule of the invention can be derived from Brassica napus varieties, such as SPE04300-075, OSE270, 0SPE487, or PyL 616 varieties.

More particularly, a nucleic acid molecule of the invention has a mutation at position 1884 (also referred to as SNP1884) of the acid nucleic sequence of SEQ ID NO 1, which causes a change in genetic codon from CCC to TCC, resulting in a substitution of a serine for a proline at position 216 relative to the wild-type amino acid sequence represented by SEQ ID NO 15.

An isolated nucleic acid molecule of the invention containing said SNP1884 mutation, resulting in a substitution of a serine for a proline at position 216, alters the functionality of the resulting FAD2 gene product, whereby the level of oleic acid is increased in plant expressing the mutant sequence, compared to the corresponding level in plant expressing the wild-type sequence.

In the framework of the invention, the term “SNP1884” refers to the single nucleotide polymorphism corresponding to said mutation at position 1884 of the nucleic acid of SEQ ID NO 1, and can refer also to the corresponding mutation in any nucleic acid molecule encoding a FAD2 protein of the invention, i.e. a FAD2 protein having a substituted amino acid at position 216 (or corresponding to position 216), and in particular having a substitution of a serine for a proline at position 216, relative to the wild-type FAD2 protein, such as the wild-type FAD2 protein represented by SEQ ID NO 11 or 15.

Any fragment of a nucleic acid molecule of the invention of at least 10, 15, 20, 25, 50, 100 or more nucleotides comprising said SNP1884 is contemplated. Examples of fragments are nucleic acid represented by SEQ ID NO 16 to 19.

Such fragments can be used as primers, as probes and/or as markers.

The nucleic acid fragments of the invention can be used as markers in plant genetic mapping and plant breeding programs.

Such markers may include restriction fragment length polymorphism (RFLP), random amplification polymorphism detection (RAPD), polymerase chain reaction (PCR) or self-sustained sequence replication (3SR) markers, for example.

Marker-assisted breeding techniques may be used to identify and follow a plant according to the invention or its progeny, also object of the invention, during the breeding process.

Marker-assisted breeding techniques may be used in addition to, or as an alternative to, other sorts of identification techniques.

An example of marker-assisted breeding is the use of PCR primers that specifically amplify a nucleic acid molecule of the invention.

The invention thereby provides methods for segregation and selection analysis of genetic crosses involving plants having nucleic acid sequences of the invention.

A method of the invention may for example involve determining the presence in a genome of particular FAD2 alleles containing at least one mutation resulting in a substitution (preferably a substitution of serine for proline) at (or corresponding to) position 216 relative to a wild type FAD2 protein, such as the wild type FAD2 protein represented by SEQ ID NO 11 or preferably 15.

Such a determination may for example be achieved with a range of techniques, such as PCR amplification, DNA fingerprinting, RNA fingerprinting, gel blotting and RFLP analysis, nuclease protection assays, sequencing of the relevant nucleic acid fragment, the generation of antibodies (monoclonal or polyclonal), or alternative methods adapted to distinguish the protein produced by the relevant alleles from other variant forms of that protein or from the wild-type.

More particularly, such fragments can be used in method of marker assisted selection for high oleic traits in plants, preferably in Brassica species, more particularly in Brassica napus varieties.

Another aspect of the present invention is related to a recombinant nucleotide sequence comprising, operably linked to a nucleotide sequence according to the invention, one or more adjacent regulatory sequence(s). Said adjacent regulatory sequence(s) is/are preferably originating from homologous organisms.

However said adjacent regulatory sequences may also be originating from heterologous organisms.

Said adjacent regulatory sequences are specific sequences such as promoters, enhancers, secretion signal sequences and/or terminators.

Another aspect of the invention is related to a vector comprising a nucleic acid molecule of the invention, possibly operably linked to one or more adjacent regulatory sequence(s) originating from homologous or from heterologous organisms.

In the present context “vector” is defined as any biochemical construct which may be used for the introduction of a nucleotide sequence (by transduction, transfection, transformation, infection, conjugation, etc.) into a cell.

Advantageously, a vector according to the invention is selected from the group consisting of plasmids (including replicative and integrative plasmids), viruses, phagemids, chromosomes, transposons, liposomes, cationic vesicles, or a mixture thereof. Said vector may already comprise one or more adjacent regulatory sequence(s), allowing the expression of said nucleic acid molecule and its transcription into a polypeptide of the invention.

The invention also relates to a FAD2 polypeptide having an amino acid substitution at (or corresponding to) position 216 relative to a wild type FAD2 protein.

Preferably, a FAD2 polypeptide of the invention comprises (or consists of) the amino acid sequence of SEQ ID NO 11 further comprising a substitution of serine for proline at position 216.

More preferably, a FAD2 polypeptide of the invention comprises (or consists of) the amino acid sequence of SEQ ID NO 15 further comprising a substitution of serine for proline at position 216.

A preferred FAD2 polypeptide of the invention comprises (or consists of) the amino acid sequence of SEQ ID NO 4.

The present invention also encompasses any fragments of a FAD2 protein of the invention having a delta-oleate desaturase activity and comprising said substitution or deletion at position 216.

Nucleic acid molecules, recombinant nucleic acid molecules, and/or vectors of the present invention are useful to transform target plants, and thereby confer altered FAD2 gene product, whereby the level of oleic acid is modified, preferably increased, in plant expressing a mutant FAD2 of the invention, compared to the corresponding level in a plant expressing the wild-type sequence.

The present invention is also related to a transformed host cell, or recombinant host cell, containing (or having incorporated) one or more of the nucleotide sequences and/or vectors according to the invention.

In the present context, a “transformed host cell” or “recombinant cell”, also referred to as “transformant”, is a cell having incorporated one or more of the nucleotide sequences and/or vectors according to the invention. The transformed host cell may be a cell in which said vector(s) and/or said nucleotide sequence(s) is/are introduced by means of genetic transformation, preferably by means of homologous recombination, or by any other well known methods used for obtaining a recombinant organism.

Any method by which the novel sequence can be incorporated into the host genome is contemplated by the present invention.

More particularly, any method by which the novel sequence can be incorporated into the host genome, and stably inherited by its progeny, is contemplated by the present invention.

A broad range of known techniques currently exist for achieving direct or indirect transformation of higher plants with exogenous nucleic acid molecules (e.g. exogenous DNA).

Transformation of plant cells can be mediated by the use of vectors. A common method of achieving transformation is the use of Agrobacterium tumefaciens to introduce a foreign gene into the target plant cell.

Plant viruses also provide a possible means for transfer of exogenous nucleic acid molecules (e.g. exogenous DNA).

Direct uptake of plant cells can also be employed. Typically, protoplasts of the target plant are placed in culture in the presence of the nucleic acid molecules to be transferred, and an agent which promotes the uptake of said nucleic acid molecules by protoplast. Useful agents in this regard are polyethylene glycol or calcium phosphate.

Alternatively, nucleic acid molecules uptake can be stimulated by electroporation. In this method, an electrical pulse is used to open temporary pores in a protoplast cell membrane, and said nucleic acid molecules in the surrounding solution are then drawn into the cell through the pores. Similarly, microinjection can be employed to deliver said nucleic acid molecules directly into a cell, and preferably directly into the nucleus of the cell.

In these techniques, transformation occurs in a plant cell in culture. Subsequent to the transformation event, plant cells can be regenerated to whole plants.

Techniques for the regeneration of mature plants from callus or protoplast culture are well known.

Alternate methods are also available which do not necessarily require the use of isolated cells, and therefore, plant regeneration techniques, to achieve transformation. These are generally referred to as “ballistic” or “particle acceleration” methods, in which nucleic acid molecules coated metal particles are propelled into plant cells by either a gunpowder charge or electrical discharge. In this manner, plant cells in culture or plant reproductive organs or cells, e.g. pollen, can be stably transformed with the nucleic acid molecules of interest.

The present invention can be applied to transformation of virtually any type of plant, monocotyledons or dicotyledons.

Suitable plants to be transformed are preferably oil producing crops, such as sunflower, soybean, cotton, corn, etc., preferably Brassica species, more preferably Brassica napus varieties.

In one aspect of the invention, a plant comprises at least one FAD2 coding sequence of the invention.

A plant of the invention can comprise a nucleic acid sequence of SEQ ID NO 1, 2 and/or 3, such as SPE04300-75.

SPE04300-75 variety is maintained as a Budapest

Treaty patent deposit with NCIMB under accession number NCIMB 41445 made Nov. 17, 2006.

Further examples of plants of the present invention comprising said SNP1884 mutation are OSE270, 0SPE487 and Py1616 varieties.

OSE270 is maintained as a Budapest Treaty patent deposit with NCIMB under accession number 41407 made on Jun. 14, 2006.

OSPE487 is maintained as a Budapest Treaty patent deposit with NCIMB under accession number NCIMB 41408 made on Jun. 14, 2006.

PyL 616 is maintained as a Budapest Treaty patent deposit with NCIMB under accession number NCIMB 41406 made on Jun. 14, 2006.

Another object of the invention is a method of producing high oleic plant lines comprising: (a) crossing a first plant with a second plant having at least one mutant FAD2 gene according to the invention, (b) obtaining seeds from the cross of step(a), (c) growing fertile plants from such seeds; (d) obtaining progeny seeds from the plants of step (c), and (e) identifying those seeds among the progeny that have high oleic acid content.

In another aspect, the invention provides a method for increasing the oleic acid content of plants, more particularly of Brassica plants, and preferably of Brassica napus plants comprising the steps of:

(a) inducing mutagenesis in at least some cells from a plant, more particularly of a Brassica plant, and preferably of a Brassica napus plant that has a oleic acid content of less than 70%;
(b) regenerating plants from at least one of said mutagenized cells;
(c) identifying and selecting regenerated plants which have a nucleic acid sequence of the invention and/or which expresses a FAD2 protein of the invention; and
(d) deriving further generations of plants from said regenerated plants.

Preferably, the seeds obtained from said plants provide an oil having an oleic acid content of more than 80wt. %, 81wt. %, 82wt. %, 83wt. %, or 84wt. %, more preferably of more than 85wt. %, 86wt. %, or even more preferably of more than 87wt. %, based upon the total weight of fatty acid present the oil.

Another object of the invention is a vegetable oil obtained from at least one plant according to the invention, which vegetable oil comprises more than (about) 80%, 81%, 82%, 83%, 84%, 85%, 86%, or 87% of oleic acid.

More particularly, a vegetable oil of the invention, obtained preferably from at least one Brassica species of the invention, more preferably from at least one Brassica napus variety according to the invention, comprises more than (about) 80%, 81%, 82%, 83%, 84%, 85%, 86%, or 87% of oleic acid. Said oil can further comprise less than (about) 4%, 3.5%, 3%, 2%, 1% or 0.5% of linolenic acid, based upon the total weight of the fatty acids present in the oil.

Preferably, said oil comprises more than (about) 80%, 81%, 82%, 83%, 84%, 85%, 86%, 87%, preferably between (about) 80% and (about) 88%, more preferably between (about) 80% and (about) 87.5% of oleic acid. Said oil can further comprise less than (about) 4%, 3.5%, 3%, 2%, 1%, or 0.5%, preferably between (about) 4% and (about) 0.4% of linolenic acid, based upon the total weight of the fatty acids present in the oil.

After an Ethyl Methane Sulfonate (EMS) treatment, a collection of high oleic winter oilseed rape varieties was grown during successive generations in the field, maintained and fixed by self-pollinations.

Their progenies were analysed for fatty acid composition using gas chromatography based analytical method, as commonly known in this area of technology.

Fatty acid composition was monitored in each generation and only material with oleic acid content higher than 75% was selected.

SPE04300-75, OSE270, OSPE487 and PyL 616 varieties exhibiting a very high oleic acid content have been obtained.

The line SPE04300-75 was thus sequenced.

Basic seed was used for the determination of fatty acid content in trials—small research trials (6 to 12 m²) or development trials (500 m²) and for the sequencing work.

### EXAMPLES

### Example 1

The seeds were grinded in a first solution consisting of methanol (800 ml), trimethyl-pentane (200 ml) and 5 g of Na OH. About 3 ml of solution was used for about 10 g of seeds (in other words about 10 to 50 seeds for 1 ml of solution).

Extraction was performed during 20 minutes and thereafter a second solution, consisting of trimethylamine (900 ml), and propanol, 2-(100 ml), was added at the same volume as the first solution.

The resulting solution was vortexed and allowed to rest until formation of an upper phase.

The upper phase was sampled and transferred into viols.

One microliter of same was injected in a gas chromatograph (Fisons from thermo-electron with a columm DB3 −30 meter with a diameter of 0.25 mm and a thickness of 25 micrometer). Running time was about 4 min.

The oleic acid content results are summarized in table 1.

TABLE 1

| | Oleic acid | | Linolenic acid |
|---|---|---|---|
| Varieties | content (wt. %) | Appreciation | content (wt. %) |
| PyL 616 | 80.5-85.1 | Very high | 2.3-2.6 |
| OSE270 | 86.4-87.5 | Very high | 3.5-3.8 |
| OSPE487 | 84.1-85.2 | Very high | 4.1-4.4 |
| SPE04300-75 | 84.4-85.6 | Very high | 4.7-5.0 |

In this example, from the seeds obtained in 2005 and 2006, the oleic acid content is higher than 80%, even higher than 84% in average, and is up to 87.5% based on the total weight of the fatty acid in the extracted oil. Besides, some varieties exhibit a low linolenic acid content, in particular a linolenic acid content equal or below 3.5%.

### Example 2

Plant materials used for sequencing are:
mutant line with higher (very high) oleic fatty acid content (compared to wild type varieties) : SPE04300-75; and
wild type varieties with normal oleic acid content: Bristol, Capitol, Vivol, Capvert and Caiman.

All these lines were grown in a growth chamber and the cotyledons and stems were collected from 7-day-old plants.

The plant tissues were freeze-dried and used for DNA extraction.

DNA was isolated with Qiagen Plant DNA kits (Qiagen INC.-USA, Valencia Calif.).

PCR was performed with TagGold protocol (AB Biosystem, Inc,).

Reaction mix includes 2.5 μl 10× buffer, 0.2 μl TagGold, 0.2 μl dNTP(25 mM), 2 μl primers (5 uM) and 10 ul DNA template (2 ng/ul) and 10.1 ul H₂O.

PCR cycles were as follows: 94° C. 5 min; 8 cycles of 94° C. 40 sec, 62° C. 40 sec, 72° C. 1 min, 94°° C. 40sec, 60° C. 40 sec, 72° C. 1 min, 94° C. 40 sec, 58 ° C. 40 sec, 72° C. 1 min, 94° C. 40 sec, 56° C. 40 sec, 72° C. 1 min; 3 cycles of 94° C. 40 sec, 55° C. 40sec, 72° C. 1 min; hold at 72° C. for 7 min.

PCR products were analyzed on 1% agarose gel.

For sequencing, 5 μl PCR products were removed to a new tube and 1 μl ExonucleaseI (1:50 dilution) and 1 μl Shrimp Alkaline Phosphatase (1:5 dilution).

The mix was incubated at 37° C. for 20 min and then 80° C. for 15 min to inactivate the enzymes.

40 μl H₂O was added and 6 μl were used as template with 1 μl sequencing primer.

Sequencing was done on 3730 DNA Analyzer (Applied Biosystems).

Sequences were assembled and aligned using SeqMan II program of the LaserGene (DNASTAR, INC, Madison. Wis.).

### Example 3

Four Brassica napus delta-12 oleate desaturase (FAD2) gene sequences, 4684997, 46399190, 8705228 and 4092878, were downloaded from Genebank (NCBI). These sequences were used as queries to blast against Monsanto sequence database.

Using the “blastn” programs (NCBI), a number of high score hits were obtained. All the hit sequences were downloaded and reassembled with the SeqmanII program (DNASTAR Inc, Madison, Wis., USA).

Two distinct transcripts were identified and designated as Fad2-1 (SEQ ID NO 9) and Fad2-2 (SEQ ID NO 13). Fad2-1 and Fad2-2 share a high sequence homology, with 97% sequence identity.

Having regard to SPE04300-75 variety, there is 96% sequence identity between the two transcripts Fad2-1 (SEQ ID NO 5) and Fad2-2 (SEQ ID NO 2).

To identify causative mutations associated with high oleic acid content in the mutant lines and their progenies, nested locus-specific primers were designed to cover the entire sequences.

The 3′ end of a primer was always located at a nucleotide that differentiated Fad2-1 from Fad2-2 except those located at 5′and 3′ ends of the consensus sequences where there was not differential nucleotide between the two genes.

The primers were also designed in such way that one amplicon would overlap with another to ensure full coverage of the entire sequence. These primers were arrayed and used to generate locus-specific amplicons on mutants and wild types. Sequencing results indicated that all the locus-specific PCR primers behaved as expected.

Sequences belonging to the same gene were assembled together using SeqManII program.

The consensus genomic sequences of the wild type Fad2-1 and Fad2-2 genes are represented respectively by SEQ ID NO 8 and 12.

Table 2 summarizes the sequence features of both Fad2-1 and Fad2-2 genes.

TABLE 2

| Features | FAD2-1 position | FAD2-2 position |
|---|---|---|
| Gene | 1-2614 | 1-2666 |
| 5′ UTR | 1-1218 | 1-1238 |
| Exon | 1-108 | 1-111 |
| Intron | 109-1213 | 112-1234 |
| Exon | 1214-2614 | 1235-2619 |
| CDS | 1218-2372 | 1239-2393 |
| 3′ UTR | 2373-2614 | 2394-2666 |

The features are based on the consensus genomic sequences from multiple reads on different genotypes.

Both Fad2-1 and Fad2-2 genes have one intron each.

The intron sizes are slightly different between the two genes. For Fad2-1, intron spans 1105 base pairs starting from position 109 to 1213, while for Fad2-2, intron consists of 1123 base pairs starting from position 112 to 1234 on the consensus sequences.

The intron is located at 5′UTR region.

Putative translation initiation codons are located at 1218 and 1239 for Fad2-1 and Fad2-2 genes, respectively.

The translation termination codons are located at 2370-2372 and 2391-2393, respectively for Fad2-1 and Fad2-2.

3′UTR sequences are 242 base pairs for Fad2-1 and 273 base pairs for Fad2-2 genes.

A point mutation was found at position 1884 (called SNP1884) of FAD2-2 gene (as represented by SEQ ID NO 1), which caused a change in genetic codon from CCC to TCC, resulting in an alternation of amino acid residue from proline to serine.

It appears that said proline at position 216 is a conformationally important amino acid and its replacement with serine is responsible for a radical change in the enzyme function in the mutant line.

1-30. (canceled)

31. An isolated nucleic acid molecule encoding a FAD2 protein having reduced desaturase activity, wherein the molecule comprises a nucleotide sequence selected from the group consisting of:
(a) a nucleotide sequence encoding a FAD2 protein having a substitution for proline at position 216 relative to a wild-type FAD2 protein;
(b) a nucleotide sequence encoding the FAD2 protein of SEQ ID NO: 4;
(c) the nucleotide sequence of SEQ ID NO: 1, its complementary form, or the RNA form thereof;
(d) the nucleotide sequence of SEQ ID NO: 2, its complementary form, or the RNA form thereof;
(e) the nucleotide sequence of SEQ ID NO: 3, its complementary form, or the RNA form thereof;
(f) a nucleotide sequence having at least 80% identity with the nucleotide sequence of SEQ ID NO: 1 wherein the codon encoding the amino acid corresponding to position 216 of SEQ ID NO: 15 encodes an amino acid other than proline;
(g) a nucleotide sequence having at least 80% identity with the nucleotide sequence of SEQ ID NO: 2 wherein the codon encoding the amino acid corresponding to position 216 of SEQ ID NO: 15 encodes an amino acid other than proline;
(h) a nucleotide sequence having at least 80% identity with the nucleotide sequence of SEQ ID NO: 3 wherein the codon encoding the amino acid corresponding to position 216 of SEQ ID NO: 15 encodes an amino acid other than proline;
(i) a nucleotide sequence fragment comprising at least 10 contiguous nucleotides of an isolated nucleic acid molecule of (c), wherein the fragment comprises a mutated codon encoding an amino acid substitution corresponding to position 216 of SEQ ID NO: 15;
(j) a nucleotide sequence fragment comprising at least 10 contiguous nucleotides of an isolated nucleic acid molecule of (d), wherein the fragment comprises a mutated codon encoding an amino acid substitution corresponding to position 216 of SEQ ID NO: 15;
(k) a nucleotide sequence fragment comprising at least 10 contiguous nucleotides of an isolated nucleic acid molecule of (e), wherein the fragment comprises a mutated codon encoding an amino acid substitution corresponding to position 216 of SEQ ID NO: 15;
(I) a nucleotide sequence that hybridizes to the nucleotide sequence of (c) under a wash stringency equivalent to 0.1×SSC, 0.15M NaCl, 0.015M trisodium citrate, at 50° C., and which encodes a FAD2 polypeptide having reduced desaturase activity as compared to a polypeptide encoded by SEQ ID NO: 15;
(m) a nucleotide sequence that hybridizes to the nucleotide sequence of (d) under a wash stringency equivalent to 0.1×SSC, 0.15M NaCl, 0.015M trisodium citrate, at 50° C., and which encodes a FAD2 polypeptide having reduced desaturase activity as compared to a polypeptide encoded by SEQ ID NO: 15; and
(n) a nucleotide sequence that hybridizes to the nucleotide sequence of (e) under a wash stringency equivalent to 0.1×SSC, 0.15M NaCl, 0.015M trisodium citrate, at 50° C., and which encodes a FAD2 polypeptide having reduced desaturase activity as compared to a polypeptide encoded by SEQ ID NO: 15.

32. A FAD2 protein comprising a polypeptide sequence selected from the group consisting of:
(a) a polypeptide encoded by the nucleic acid molecule of claim 31;
(b) a polypeptide encoding a FAD2 protein having an amino acid substitution for the amino acid corresponding to position 216 of a wild-type FAD2 protein;
(c) a polypeptide comprising SEQ ID NO: 11, wherein SEQ ID NO: 11 comprises an amino acid substitution at position 216;
(d) a polypeptide comprising SEQ ID NO: 15, wherein SEQ ID NO: 15 comprises an amino acid substitution at position 216; and
(e) a polypeptide having the amino acid sequence of SEQ ID NO: 4;
wherein said FAD2 protein has reduced desaturase activity relative to a wild-type FAD2 protein.

33. The isolated nucleic acid molecule according to claim 31 wherein the substitution at or corresponding to position 216 is substitution with a non-ionizable polar amino acid.

34. The isolated nucleic acid molecule according to claim 33 wherein the non-ionizable polar amino acid is selected from the group consisting of threonine, asparagine, glutamine, cysteine, tyrosine, and serine.

35. A vector comprising a nucleic acid molecule according to claim 31.

36. A host cell comprising a nucleic acid molecule according to claim 31.

37. A plant stably transformed with a vector of claim 35.

38. The plant of claim 37, wherein the plant is selected from the group consisting of sunflower, soybean, cotton, corn, and rapeseed.

39. A plant, plant part, or seed containing the nucleic acid molecule of claim 31.

40. A plant, plant part, or seed containing the protein of claim 32.

41. A method of enhancing the oleic acid content in a plant comprising transforming a plant with the vector of claim 35.

42. A method of producing high oleic plant lines, the method comprising:
(a) inducing mutagenesis in a cell from a plant that has an oleic acid content of less than 70%;
(b) regenerating a plant from said mutagenized cell;
(c) selecting regenerated plants which have a nucleic acid sequence of claim 31; and
(d) deriving further generations of plants from said regenerated plants.

43. The method according to claim 42, wherein the cell from a plant is from a Brassica plant.

44. The method according to claim 43, wherein the Brassica plant is a Brassica napus plant.

45. The method according to claim 42, wherein the mutagenesis is induced by ethyl methane sulfonante treatment.

46. A plant obtained by the method of claim 45.

47. A method of producing high oleic plant lines, the method comprising:
(a) crossing a first plant of claim 37 with a second plant,
(b) obtaining seeds from the cross of step (a),
(c) growing fertile plants from the seeds,
(d) obtaining progeny seeds from the plants of step(c), and
(e) identifying those seeds among the progeny that have high oleic acid content.

48. A vegetable oil obtained from the plant, plant part, or seed of claim 39, said oil comprising an oleic acid content selected from the group consisting of more than about 80%, about 84%, and about 85% of oleic acid based upon the total weight of the fatty acids present in the oil.

49. The vegetable oil according to claim 48 further comprising a linolenic acid content selected from the group consisting of less than about 4%, 3.5%, 3%, 2%, 1% and 0.5% of linolenic acid based upon the total weight of the fatty acids present in the oil.

50. A food or a feed product comprising and/or prepared with the vegetable oil of claim 48.

51. A food or a feed product comprising and/or prepared with the plant, plant part, or a seed of claim 38.

52. The food or feed product of claim 51, wherein the food or feed product is vegetable meal.


SEQUENCE LISTING (20 sequences)

SEQ ID NO: 1 - DNA, 2666 bp, Brassica napus
gagaagagagagagagagagagagagagagagtgagtttgaggaggagcttcttcgtagggttcatcgttattaacgttaaatcttcaccccctacgtcagccagctcaaggtccctttcttcttccatttcttttcattctacgttgttttcaatcttatgaaactttctggtctgtgcttttcttatcgcttttctattctatctatcatttttgcatttcagtcgatttaattctagatctgttaatattaaactatagatctgttcttgattctctgttttcatgtgtgaaatctgatgctgtattaatctgattatattgtctataccgtggagaatatcaaatgttgcattttcatttgtccgaatacaaagtgtttgactttcaatcgtttttaattatatatatatatatattttttgatgggttggtggagttgaaaaatcaccatagcagtctcacgtcctggttttagaaatatcctattcaaaattatatatttgtttacttgttttagatctggacctgagacatataagtacctatttgttgaatctttgggtaaaaacttatgtctctgggtaaaatttgctgggagatttgaccgattcctattggctcttgattctgtagttacgtaatacatgaaaaagtttcatttggcctatgctcacttcatgcttataaacgttttcttgcaaattaattggattagatgttatttcatagattcagtcattcagatacaatggagttgcatgaagaaaataatagaattcgtgacagtaaaaaagattgtatttttgtttgtttgtttatgtttaaaagtctatatgttgacaatagagttgctctcaactgtttcatttagcttctttttttgtcaagttgcttattcttagagacattgtgattatgacttgtcttctttaacgtagtttagtaataaaagacgaaagaaattgatatccacaagaaagagatgtgagctgtagcgtatcaaatctcgttcatttactagtagtattctcaacgctatcgtttatttatttttctttcgttggtttgccactatatgccacttctctcctctttgtcccacgtactatccattttttttgtggtagtccattttcttgtaacttataataacgtaactctgaatcttttgtctgtagattaatttgttggtttaattaacttttaagtctttgcttttggcttatgcagaaacatgggtgcaggtggaagaatgcaagtgtctcctccctccaagaagtctgaaaccgacaccatcaagcgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggtttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgcttccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaggtcttccacaatattaccgacacgcacgtggcgcatcatctgttctccacgatgccgcattatcacgcgatggaagctaccaaggcgataaagccgatactgggagagtattatcagttcgatgggacgccggtggttaaggcgatgtggagggaggcgaaggagtgtatctatgtggaaccggacaggcaaggtgagaagaaaggtgtgttctggtacaacaataagttatgaggatatgatgatggtgaaagaacaaagaagatattgtcacgaacctttctcttgctgtctctggtcgtctttgttttaagaagctatgttttcgtttcaataatcttaactatccattttgttgtgttttctgacattttggctaaaattatgtgatgttggaagttagtgtctaaaatgtcttgtgtctgtattgttcttcttctcatcgctgttatgtttgggatcgttgaaatgtgactttcggactagtgaactcttggttctcgaact

SEQ ID NO: 2 - DNA, 1553 bp, Brassica napus
gagacagattcattaccaaagagatagagaaagagagagagagagagagagagagagagtgagtttgaggaggagcttcttcgtagggttcatcgttattaacgttaaatcttcaccccctacgtcagccagctcaagaaacatgggtgcaggtggaagaatgcaagtgtctcctccctccaagaagtctgaaaccgacaccatcaagcgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgcttccatctacaacgaccgcgagcgtctccagatatacatctccgacgctggcatcctcgccgtctgctacggtctcttccgttacgccgccgsscagggagtggcctcgatggtctgcttctacggagtcccgcttctgattgtcaatggtttcctcgtgttgatcacttacttgcagcacacgcatccttccctgcctcactacgattcgtccgagtgggattggttsaggggagctttggctaccgttgacagagactacggaatcttgaacaaggtcttccacaatattaccgacacgcacgtggcscatcatcygttctccacgatgccgcattatcacgcgatggaagctaccaaggcgataaagccgatactgggagagtattatcagttcgatgggacgccggtggttaaggcgatgtggagggaggcgaaggagtgtatctatgtggaaccggacaggcaaggtgagaagaaaggtgtgttctggtacaacaataagttatgaggatrraagaaactgaacctttctcttcctatgattgtctttgtttaagaagctatgtttctgtttcaataatcttaattatccattttgttgtgttttctgacattttggctaaaattatgtgatgttggaagttagtgtctaaaatgtcttgtgtctgtattgttcttcttctcatcgctgttatgtttgggatcgttgaaatgtgactttcggactagtgaactcttgttctcgaactaaaaaaaaaaaaaaa

SEQ ID NO: 3 - DNA, 1155 bp, Brassica napus
  CDS (1)..(1155)
atgggtgcaggtggaagaatgcaagtgtctcctccctccaagaagtctgaaaccgacaccatcaagcgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgcttccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaggtcttccacaatattaccgacacgcacgtggcgcatcatctgttctccacgatgccgcattatcacgcgatggaagctaccaaggcgataaagccgatactgggagagtattatcagttcgatgggacgccggtggttaaggcgatgtggagggaggcgaaggagtgtatctatgtggaaccggacaggcaaggtgagaagaaaggtgtgttctggtacaacaataagttatga

SEQ ID NO: 4 - protein, 384 aa, Brassica napus
MGAGGRMQVSPPSKKSETDTIKRVPCETPPFTVGELKKAIPPHCFKRSIPRSFSYLIWDIIIASCFYYVATTYFPLLPHPLSYFAWPLYWACQGCVLTGVWVIAHECGHHAFSDYQWLDDTVGLIFHSFLLVPYFSWKYSHRRHHSNTGSLERDEVFVPKKKSDIKWYGKYLNNPLGRTVMLTVQFTLGWPLYLAFNVSGRPYDGGFACHFHPNASIYNDRERLQIYISDAGILAVCYGLFRYAAAQGVASMVCFYGVPLLIVNGFLVLITYLQHTHPSLPHYDSSEWDWLRGALATVDRDYGILNKVFHNITDTHVAHHLFSTMPHYHAMEATKAIKPILGEYYQFDGTPVVKAMWREAKECIYVEPDRQGEKKGVFWYNNKL

SEQ ID NO: 5 - DNA, 1561 bp, Brassica napus
gagaaccagagagattcattaccaaagagatagagagagagagaaagagaggagacagagagagagtttgaggaggagcttcttcgtagggttcatcgttattaacgttaaatcttcatccccccctacgtcagccagctcaagaaacatgggtgcaggtggaagaatgcaagtgtctcctccctccaaaaagtctgaaaccgacaacatcaagcgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccagggctgcgtcctaaccggcgtctgggtcatagcccacgagtgcggccaccacgccttcagcgactaccagtggctggacgacaccgtcggcctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggcctttgtacttagccttcaacgtctcggggagaccttacgacggcggcttcgcttgccatttccaccccaacgctcccatctacaacgaccgtgagcgtctccagatatacatctccgacgctggcatcctcgccgtctgctacggtctctaccgctacgctgctgtccaaggagttgcctcgatggtctgcttctacggagttcctcttctgattgtcaacgggttcttagttttgatcacttacttgcagcacacgcatccttccctgcctcactatgactcgtctgagtgggattggttgaggggagctttggccaccgttgacagagactacggaatcttgaacaaggtcttccacaatatcacggacacgcacgtggcgcatcacctgttctcgaccatgccgcattatcatgcgatggaagctacgaaggcgataaagccgatactgggagagtattatcagttcgatgggacgccggtggttaaggcgatgtggagggaggcgaaggagtgtatctatgtggaaccggacaggcaaggtgagaagaaaggtgtgttctggtacaacaataagttatgaagcaaagaagaaactgaacctttctcwtcctatgattgtctttgtttaagaagctatgtttctgtttcaataatctttaattatccattttgttgtgttttctgacattttggctaaaattatgtgatgttggaagttagtgtctaaaatgtcttgtgtctgtattgttcttcttctcatcgctgttatgtttgggatcgttgaaatgtgactttcggactagtgaactcttgttctcgaactaaaaaaaaaaaaaaa

SEQ ID NO: 6 - DNA, 1155 bp, Brassica napus
  CDS (1)..(1155)
atgggtgcaggtggaagaatgcaagtgtctcctccctccaaaaagtctgaaaccgacaacatcaagcgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccagggctgcgtcctaaccggcgtctgggtcatagcccacgagtgcggccaccacgccttcagcgactaccagtggctggacgacaccgtcggcctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggcctttgtacttagccttcaacgtctcggggagaccttacgacggcggcttcgcttgccatttccaccccaacgctcccatctacaacgaccgtgagcgtctccagatatacatctccgacgctggcatcctcgccgtctgctacggtctctaccgctacgctgctgtccaaggagttgcctcgatggtctgcttctacggagttcctcttctgattgtcaacgggttcttagttttgatcacttacttgcagcacacgcatccttccctgcctcactatgactcgtctgagtgggattggttgaggggagctttggccaccgttgacagagactacggaatcttgaacaaggtcttccacaatatcacggacacgcacgtggcgcatcacctgttctcgaccatgccgcattatcacgcgatggaagctacgaaggcgataaagccgatactgggagagtattatcagttcgatgggacgccggtggttaaggcgatgtggagggaggcgaaggagtgtatctatgtggaaccggacaggcaaggtgagaagaaaggtgtgttctggtacaacaataagttatga

SEQ ID NO: 7 - protein, 384 aa, Brassica napus
MGAGGRMQVSPPSKKSETDNIKRVPCETPPFTVGELKKAIPPHCFKRSIPRSFSYLIWDIIIASCFYYVATTYFPLLPHPLSYFAWPLYWACQGCVLTGVWVIAHECGHHAFSDYQWLDDTVGLIFHSFLLVPYFSWKYSHRRHHSNTGSLERDEVFVPKKKSDIKWYGKYLNNPLGRTVMLTVQFTLGWPLYLAFNVSGRPYDGGFACHFHPNAPIYNDRERLQIYISDAGILAVCYGLYRYAAVQGVASMVCFYGVPLLIVNGFLVLITYLQHTHPSLPHYDSSEWDWLRGALATVDRDYGILNKVFHNITDTHVAHHLFSTMPHYHAMEATKAIKPILGEYYQFDGTPVVKAMWREAKECIYVEPDRQGEKKGVFWYNNKL

SEQ ID NO: 8 - DNA, 2614 bp, Brassica napus
agagagagaagagaggagacagagagagagtttgaggaggagcttcttcgtagggttcatcgttattaacgttaaatcttcatccccccctacgtcagccagctcaaggtccctttcttcttccatttcttctcatttttacgttgttttcaatcttggtctgttcttttcttatcgcttttctgttctatctatcatttttgcatttcagtcgatttatttctagatctgttaatatttattgcattaaactatagatctggtcttgattctctgttttcatgtgtgaaatcttgatgctgtcttaaccattaatctgattatattgtctataccgtggagaatatgaaatgttgcattttcatttgtccgaatacaaactgtttgactttcaatcgtttttaaaattatatatatatttttgatgggttggtggagttgaaaaatcaccatagcagtctcacgtcctggtcttagaaatatccttcctattcaaagttatatatatttgtttacttttgttttagatctggacctgagacatgtaagtacatatttgttgaatctttgggtaaaaaacttatgtctctgggtaaaatttgctgagagatttgaccgattcctattggctctggattctgtagttacctaatacatgaaaaagtttcatttggcctatgctcacttcatgcttataaactttttcttgcaaattaattggattagatgctccttcatagattcagatgcaatagatttgcatgaagaaaataataggattcatgatagtaaaaaagattgtatttttgtttgtttgtttatgtttaaaagtctatatgttgacaatagagttgctatcaactgtttcatttaggtttatgtttttgtcaagttgcttattctaagagacattgtgattatgacttgtcttctctaacgtagtttagtaataaaagacgaaagaaattgatatccacaagaaagagatgtaagctgtaacgtatcaaatctcattaataactagtagtattctcaacgctatcgtttatttctttctttggtttgccactatatgccgcttctctgctcttttatcccacgtactatccattttttttgtggtagtccatttttttgaaactttaataacgtaacactgaatattaatttgttggtttaattaactttgagtctttgcttttggtttatgcagaaacatgggtgcaggtggaagaatgcaagtgtctcctccctccaaaaagtctgaaaccgacaacatcaagcgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccagggctgcgtcctaaccggcgtctgggtcatagcccacgagtgcggccaccacgccttcagcgactaccagtggctggacgacaccgtcggcctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggcctttgtacttagccttcaacgtctcggggagaccttacgacggcggcttcgcttgccatttccaccccaacgctcccatctacaacgaccgtgagcgtctccagatatacatctccgacgctggcatcctcgccgtctgctacggtctctaccgctacgctgctgtccaaggagttgcctcgatggtctgcttctacggagttcctcttctgattgtcaacgggttcttagttttgatcacttacttgcagcacacgcatccttccctgcctcactatgactcgtctgagtgggattggttgaggggagctttggccaccgttgacagagactacggaatcttgaacaaggtcttccacaatatcacggacacgcacgtggcgcatcacctgttctcgaccatgccgcattatcacgcgatggaagctacgaaggcgataaagccgatactgggagagtattatcagttcgatgggacgccggtggttaaggcgatgtggagggaggcgaaggagtgtatctatgtggaaccggacaggcaaggtgagaagaaaggtgtgttctggtacaacaataagttatgaagcaaagaagaaactgaacctttctcatctatgattgtctttgttttaagaagctatgtttctgtttcaataatctttaattatccattttgttgtgttttctgacattttggctaaaattatgtgatgttggaagttagtgtctaaaatgtcttgtgtctgtattgttcttcttctcatcgctgttatgtttgggatcgttgaaatgtgactttcggactagtgaatcttgttctcgaact

SEQ ID NO: 9 - DNA, 1561 bp, Brassica napus; identical to SEQ ID NO: 5

SEQ ID NO: 10 - DNA, 1155 bp, Brassica napus; identical to SEQ ID NO: 6
  CDS (1)..(1155)

SEQ ID NO: 11 - protein, 384 aa, Brassica napus; identical to SEQ ID NO: 7

SEQ ID NO: 12 - DNA, 2666 bp, Brassica napus
gagaagagagagagagagagagagagagagagtgagtttgaggaggagcttcttcgtagggttcatcgttattaacgttaaatcttcaccccctacgtcagccagctcaaggtccctttcttcttccatttcttttcattctacgttgttttcaatcttatgaaactttctggtctgtgcttttcttatcgcttttctattctatctatcatttttgcatttcagtcgatttaattctagatctgttaatattaaactatagatctgttcttgattctctgttttcatgtgtgaaatctgatgctgtattaatctgattatattgtctataccgtggagaatatcaaatgttgcattttcatttgtccgaatacaaagtgtttgactttcaatcgtttttaattatatatatatatatattttttgatgggttggtggagttgaaaaatcaccatagcagtctcacgtcctggttttagaaatatcctattcaaaattatatatttgtttacttgttttagatctggacctgagacatataagtacctatttgttgaatctttgggtaaaaacttatgtctctgggtaaaatttgctgggagatttgaccgattcctattggctcttgattctgtagttacgtaatacatgaaaaagtttcatttggcctatgctcacttcatgcttataaacgttttcttgcaaattaattggattagatgttatttcatagattcagtcattcagatacaatggagttgcatgaagaaaataatagaattcgtgacagtaaaaaagattgtatttttgtttgtttgtttatgtttaaaagtctatatgttgacaatagagttgctctcaactgtttcatttagcttctttttttgtcaagttgcttattcttagagacattgtgattatgacttgtcttctttaacgtagtttagtaataaaagacgaaagaaattgatatccacaagaaagagatgtgagctgtagcgtatcaaatctcgttcatttactagtagtattctcaacgctatcgtttatttatttttctttcgttggtttgccactatatgccacttctctcctctttgtcccacgtactatccattttttttgtggtagtccattttcttgtaacttataataacgtaactctgaatcttttgtctgtagattaatttgttggtttaattaacttttaagtctttgcttttggcttatgcagaaacatgggtgcaggtggaagaatgcaagtgtctcctccctccaagaagtctgaaaccgacaccatcaagcgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggtttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaggtcttccacaatattaccgacacgcacgtggcgcatcatctgttctccacgatgccgcattatcacgcgatggaagctaccaaggcgataaagccgatactgggagagtattatcagttcgatgggacgccggtggttaaggcgatgtggagggaggcgaaggagtgtatctatgtggaaccggacaggcaaggtgagaagaaaggtgtgttctggtacaacaataagttatgaggatatgatgatggtgaaagaacaaagaagatattgtcacgaacctttctcttgctgtctctggtcgtctttgttttaagaagctatgttttcgtttcaataatcttaactatccattttgttgtgttttctgacattttggctaaaattatgtgatgttggaagttagtgtctaaaatgtcttgtgtctgtattgttcttcttctcatcgctgttatgtttgggatcgttgaaatgtgactttcggactagtgaactcttggttctcgaact

SEQ ID NO: 13 - DNA, 1553 bp, Brassica napus
gagacagattcattaccaaagagatagagaaagagagagagagagagagagagagagagtgagtttgaggaggagcttcttcgtagggttcatcgttattaacgttaaatcttcaccccctacgtcagccagctcaagaaacatgggtgcaggtggaagaatgcaagtgtctcctccctccaagaagtctgaaaccgacaccatcaagcgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgsscagggagtggcctcgatggtctgcttctacggagtcccgcttctgattgtcaatggtttcctcgtgttgatcacttacttgcagcacacgcatccttccctgcctcactacgattcgtccgagtgggattggttsaggggagctttggctaccgttgacagagactacggaatcttgaacaaggtcttccacaatattaccgacacgcacgtggcscatcatcygttctccacgatgccgcattatcacgcgatggaagctaccaaggcgataaagccgatactgggagagtattatcagttcgatgggacgccggtggttaaggcgatgtggagggaggcgaaggagtgtatctatgtggaaccggacaggcaaggtgagaagaaaggtgtgttctggtacaacaataagttatgaggatrraagaaactgaacctttctcttcctatgattgtctttgtttaagaagctatgtttctgtttcaataatcttaattatccattttgttgtgttttctgacattttggctaaaattatgtgatgttggaagttagtgtctaaaatgtcttgtgtctgtattgttcttcttctcatcgctgttatgtttgggatcgttgaaatgtgactttcggactagtgaactcttgttctcgaactaaaaaaaaaaaaaaa

SEQ ID NO: 14 - DNA, 1155 bp, Brassica napus
  CDS (1)..(1155)
atgggtgcaggtggaagaatgcaagtgtctcctccctccaagaagtctgaaaccgacaccatcaagcgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaggtcttccacaatattaccgacacgcacgtggcgcatcatctgttctccacgatgccgcattatcacgcgatggaagctaccaaggcgataaagccgatactgggagagtattatcagttcgatgggacgccggtggttaaggcgatgtggagggaggcgaaggagtgtatctatgtggaaccggacaggcaaggtgagaagaaaggtgtgttctggtacaacaataagttatga

SEQ ID NO: 15 - protein, 384 aa, Brassica napus
MGAGGRMQVSPPSKKSETDTIKRVPCETPPFTVGELKKAIPPHCFKRSIPRSFSYLIWDIIIASCFYYVATTYFPLLPHPLSYFAWPLYWACQGCVLTGVWVIAHECGHHAFSDYQWLDDTVGLIFHSFLLVPYFSWKYSHRRHHSNTGSLERDEVFVPKKKSDIKWYGKYLNNPLGRTVMLTVQFTLGWPLYLAFNVSGRPYDGGFACHFHPNAPIYNDRERLQIYISDAGILAVCYGLFRYAAAQGVASMVCFYGVPLLIVNGFLVLITYLQHTHPSLPHYDSSEWDWLRGALATVDRDYGILNKVFHNITDTHVAHHLFSTMPHYHAMEATKAIKPILGEYYQFDGTPVVKAMWREAKECIYVEPDRQGEKKGVFWYNNKL

SEQ ID NO: 16 - DNA, 18 bp, Brassica napus
gccttcaacgtctcggga

SEQ ID NO: 17 - DNA, 24 bp, Brassica napus
gagatgtatatctggagacgctcg

SEQ ID NO: 18 - DNA, 15 bp, Brassica napus
ccaacgctcccatct

SEQ ID NO: 19 - DNA, 15 bp, Brassica napus
ccaacgcttccatct

SEQ ID NO: 20 - DNA, 2614 bp, Brassica napus
  misc_feature (406)..(407): n is a, c, g, or t
agagagagaagagaggagacagagagagagtttgaggaggagcttcttcgtagggttcatcgttattaacgttaaatcttcatccccccctacgtcagccagctcaaggtccctttcttcttccatttcttctcatttttacgttgttttcaatcttggtctgttcttttcttatcgcttttctgttctatctatcatttttgcatttcagtcgatttaattctagatctgttaatatttattgcattaaactatagatctggtctcgattctctgttttcatgtgtgaaatcttgatgctgtctttaccattaatctgcttatattgtatataccgtggagaatatgaaatgttgcattttcatttgtccgaatacaaactgtttgacttccaatcgtttttaannttatatatatnnttttgatgggttggtggagttgaaaaatcaccatagcagtctcacgtcctggttttagaaatatccttcctattcaaagtnntatatatttgtttacttttgttttagatctggacctgagacatgtaagtacatatttgttgaatctttgggtnaaaantttatgtctctgggtaaaatttgctgagagatttgaccgattcctattggctctggattctgtannnnnnnnntacatgaaaaagtttcattnggcctatgctcacgtcatgcttacaaacttttctttgcaaattaattcgattagatgctccttcatagattcagatgcaatagatttgcatgaagaaaataataggattcatgatagtnaaaaagattgtacatttttttgtttgtttatgtttaaaagtctatatgttgacaatagagttgctatcaactgtttcatttaggtttttgtttttctcaagttgcttattcttagagacattgtgattatgacttgtcgtctttaacgtagtttagtaataaaagacgaaagaaattgatatccacaagaaagagatgtgagctgtagcgtatcaaatctcattaataactagtagtattctcaacgctatcgtttatttctttctttggtttgccactatatgccgcttctctgctcntttatcccacgtactatccattttttttgtggtagtccatttttttgaaactttaataacgtaacactgaatattaatttgttggtttaattaactttgagtctttgcttttggtttatgcagaaacatgggtgcaggtggaagaatgcaagtgtctcctccctccaaaaagtctgaaaccgacaacatcaagcgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccagggctgcgtcctaaccggcgtctgggtcatagcccacgagtgcggccaccacgccttcagcgactaccagtggctggacgacaccgtcggcctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggcctttgtacttagccttcaacgtctcggggagaccttacgacggcggcttcgcttgccatttccaccccaacgctcccatctacaacgaccgtgagcgtctccagatatacatctccgacgctggcatcctcgccgtctgctacggtctctaccgctacgctgctgtccaaggagttgcctcgatggtctgcttctacggagttcctcttctgattgtcaacgggttcttagttttgatcacttacttgcagcacacgcatccttccctgcctcactatgactcgtctgagtgggattggttgaggggagctttggccaccgttgacagagactacggaatcttgaacaaggtcttccacaatatcacggacacgcacgtggcgcatcacctgttctcgaccatgccgcattatcatgcgatggaagctacgaaggcgataaagccgatactgggagagtattatcagttcgatgggacgccggtggttaaggcgatgtggagggaggcgaaggagtgtatctatgtggaaccggacaggcaaggtgagaagaaaggtgtgttctggtacaacaataagttatgaagcaaagaagaaactgaacctttctcttctatgattgtctttgntttaagaagctatgtttctgtttcaataatcnttaattatccattttgttgtgttttctgacattttggctaaaattatgtgatgttggaagttagtgtctaaaatgtcttgtgtctgtattgttcttcttctcatcgctgttatgtttgggatcgttgaaatgtgactttcggactagtgaatcttgttctcgaact